(19) United States
(12) Patent Application Publication
(10) Pub. No.: US 2013/0218053 A1
(43) Pub. Date: Aug. 22, 2013

(54) SYSTEM COMPRISED OF SENSORS, COMMUNICATIONS, PROCESSING AND INFERENCE ON SERVERS AND OTHER DEVICES

(71) Applicants: William J. Kaiser, Los Angeles, CA (US); Bruce H. Dobkin, Los Angeles, CA (US); Majid Sarrafzadeh, Anaheim Hills, CA (US); Greg Pottie, Los Angeles, CA (US); Maxim A. Batalin, San Diego, CA (US); Xiaoyu Xu, Los Angeles, CA (US)

(72) Inventors: William J. Kaiser, Los Angeles, CA (US); Bruce H. Dobkin, Los Angeles, CA (US); Majid Sarrafzadeh, Anaheim Hills, CA (US); Greg Pottie, Los Angeles, CA (US); Maxim A. Batalin, San Diego, CA (US); Xiaoyu Xu, Los Angeles, CA (US)

(73) Assignee: THE REGENTS OF THE UNIVERSITY OF CALIFORNIA, Oakland, CA (US)

(21) Appl. No.: 13/809,399

(22) PCT Filed: Jul. 8, 2011
(86) PCT No.: PCT/US11/43397

§ 371 Date: Apr. 30, 2013

Related U.S. Application Data
(60) Provisional application No. 61/363,115, filed on Jul. 9, 2010.

(51) Int. Cl. A61B 5/11; A61B 5/00

(52) U.S. Cl. CPC A61B 5/11; A61B 5/7257; A61B 5/0004; A61B 5/6801
USPC 600/595

(57) ABSTRACT

A system for monitoring patient activity comprising: at least one measurement device configured to provide data related to a patient's physical activity; and a server configured to make an inference regarding the patient's physical activity based on data provided by the at least one measurement device. In some embodiments, the inference is a determination of a type of physical activity. In some embodiments, the measurement device is configured to be worn by the patient or carried in the patient's pocket. In some embodiments, two or more measurement devices are used. In some embodiments, the server is remotely located from the measurement device. In some embodiments, the server is configured to archive and retrieve the data provided by the measurement device and the inferences.

### CROSS-REFERENCE TO RELATED APPLICATIONS

This application claims priority to co-pending U.S. Provisional Application Ser. No. 61/363,115, filed Jul. 9, 2010, entitled “SYSTEM COMPRISED OF SENSORS, COMMUNICATIONS, PROCESSING AND INFERENCE ON SERVERS AND OTHER DEVICES,” which is hereby incorporated by reference as if set forth herein.

### FIELD OF THE INVENTION

The present invention relates to the monitoring of physical activities of patients. More specifically, the present invention relates to a method of and system for monitoring the physical activities of patients using measuring devices and a remote server.

### BACKGROUND OF THE INVENTION

Most rehabilitation, beyond the first few months following stroke, spinal cord injury (SCI), and brain injury (BI) from trauma and tumors, is, at best, home based under intermittent supervision by therapists or the family. Rehabilitative care for diseases that accrue disability over time, such as multiple sclerosis (MS), Parkinson's, dementias, neuromuscular diseases, polyneuropathies, and cerebral palsy (CP), may be even less structured when attempts are made to maintain or increase motor skills and improve strength and fitness. For a home-based therapeutic program that aims to lessen impairments and disabilities, physicians and therapists are usually unable to determine a patient's progress in terms of actual time and effort spent on prescribed activities, including the number of repetitions, energy cost, and the quality of practiced movements. Self-reports as a monitoring tool may not be accurate. The ability of patients to self-monitor is probably even less reliable when more technical information is given about practice parameters, such as advice about ways to reduce gait deviations that impede balance or raise the energy cost of walking.

Most randomized clinical trials (RCTs) reveal that counseling about exercise and training of skills fails to improve outcomes. Counseling about physical activity has had modest benefits on elderly sedentary people, as well as on risk factor reduction for diabetes, hypertension, hypercholesterolemia, and obesity, but general effectiveness is uncertain. For neurologic diseases such as stroke, the best and most cost effective way of increasing physical activity has not been found. For example, a randomized, international trial of 314 subjects, who were verbally instructed and encouraged in a detailed training program before inpatient discharge and reinforced at 5 follow-up visits over 24 months, found no greater physical activity at each visit than the controls who had follow-up visits but no physical activity instructions. The intervention also had no effect on recurrent stroke, MI, mortality, falls, and fractures. The trial also confirmed that post-stroke outpatients demonstrate very low levels of physical activity. More intensive strategies appear necessary to promote physical activity when a clinic or home trainer is not available. Some supervised training may be almost as good as greater supervision of exercise, although the outcomes tend toward statistically significant, but clinically modest gains. The efficacy of highly supervised and rather intensive training to improve balance, walking speed, fitness, or use of the affected upper extremity after stroke has been far more convincing.

Clinical trials of physical interventions to lessen impairments and disabilities are also burdened by the inability to monitor formal practice of motor skills, unless they devote a large portion of their budget to outpatient travel and clinic facilities. For example, the ongoing NIH-funded Locomotor Experience Applied Post Stroke Trial (LEAPS) has had to spend $1M per site to manage 36 sessions per subject of locomotor or mat exercise training by therapists, which amounts to at least half the budget of $12M. Although not fully applicable to that trial, many types of interventions could be designed to be monitored by the Personal Activity Montiors (PAMs) of the present invention (which will be discussed below) without frequent hands-on supervision, and thus reduce the cost of RCTs. In addition, the total amount of practice by subjects in each arm of an RCT is difficult to assess beyond what can be tallied during formal interactions with a therapist. Researchers cannot readily control for or measure what subjects practice in between formally directed therapies or from the time the intervention stops until final outcome measures are performed. This variation in subject activity and intensity of informal practice can confound the interpretation of gains attributed to the type and dose of the intervention itself.

Perhaps most important, clinical researchers have few options for measuring outcomes of their interventions in an environment other than a laboratory. Laboratory measures in most trials serve as surrogates for the outcomes that are most meaningful to the investigators, and perhaps to their subjects. For example, the walking speed on a flat tiled floor for 50 feet is a reproducible measure that subsumes many factors such as strength, motor control, gait efficiency, and potential for indoor and outdoor mobility. Walking at a faster speed after a therapy intervention, however, says little about whether a subject actually walks faster and further in the home and community or recovers the ability to walk efficiently and safely crossing streets, attending social events, and shopping for food. In the multi-center, NIH-funded Spinal Cord Injury Locomotor Trial (SCILT), for example, the inventors of the present invention were unable to detect differences in real-world activities based on the physical functioning scale of the Medical Outcomes Study SF-36, which draws upon the patient's perspective, in relation to each quartile of the final walking speeds of participants. This standard quality of life (QoL) scale may not have offered enough precision about how much and how well the walking patients performed at home and in the community. The clinical meaningfulness of a gain in the primary outcome of an RCT for a continuous variable like walking speed may also be moot when compared to the range of mobility activities that subjects value and aim to perform.

Researchers have sought direct, ecologically valid measures of upper and lower extremity activities. Inexpensive interval and ratio measures of real-world functioning could offer high face validity for outcomes valued by investigators and their subjects. Indeed, the extent to which the limitations of existing rating scales are to blame for the failure of clinical trials to deliver treatments, while unknown, is a source of discomfort for all trialists. With better tools, trialists could overcome some of the major barriers to the optimal design of clinical trials of rehabilitation interventions and more reliably and efficiently develop evidence-based practices. Ideally, these measures of motor function would serve both to monitor a therapy and to obtain outcomes captured repeatedly over days in ecologically meaningful settings, rather than briefly sample activity in a laboratory. In addition, optimal activity-based outcome measures would be agnostic, in the sense that they would not be disease-centric. Rather, the tools would help integrate the domains of sensorimotor impairment, disability, activity, and participation, regardless of pathology.

### SUMMARY OF THE INVENTION

Most embedded systems are designed for a single purpose, with a closed architecture. Medical monitors in particular are seldom designed for multiple user communities, typically being targeted either only at the patient or the medical professional. Either no data goes to the medical professional, or it is presented in only a limited number of ways. The PAM system of the present invention presents a new paradigm for personalized health care. It is an end-to-end modular system in which the patient, family members, nurses, physicians, and medical researchers can all access data via interfaces that can be specialized to their very different needs. Low cost and robust physical monitoring devices are paired with server systems enabling sophisticated and flexible analysis of data. A layered processing architecture enables high-priority events to be quickly flagged, and the data to be searched and processed to meet differing goals over time. A common look and feel coupled with built-in inference engines enable new monitoring devices to be added, expanding the scope of applications, while minimizing re-training in how to effectively use the system. The system provides patients and caregivers with unprecedented feedback concerning compliance with therapy, effectiveness of therapy, and provides quantitative records that enable both improved individual care regimes and low-cost studies across large populations.

In some embodiments, the present invention is an end-to-end system comprised of sophisticated, inexpensive sensors together with communications means and processing in servers and other devices that provide reliable inferences and convenient user interfaces concerning the types, quantity, and quality of the physical activities of patients in their homes and communities. This system enables laboratory quality data to be made available from subjects as they carry out a prescribed set of exercises at home and as they interact naturally with their environment, while also providing feedback that is directly understandable by patients and non-expert caregivers. This data and feedback enhances research methods needed to monitor compliance with prescribed rehabilitation interventions and measure outcomes for clinical trials in neurologic rehabilitation. In some embodiments, the sensors, also referred to as PAMs, are wireless. In some embodiments, the sensors include triaxial accelerometers that detect limb and truncal movement in 3 planes. In some embodiments, the sensors comprise or are integrated with microgyroscopes to detect rotational movements, global positioning satellite (GPS) data to distinguish indoor from outdoor activity, voice recorders to allow personal notations about activities, and/or heart rate monitors for cardiovascular information. The present invention allows for additional sensors to be easily integrated into the system. Continued development and application of this technology offers many opportunities to improve the design of clinical trials and manage the rehabilitation of individual patients, leading to both cost reduction and improved clinical outcome.

The PAM system of the present invention is a complete architecture that provides a fundamental advance over conventional activity monitoring systems. In some embodiments, the PAM signal processing and state classification system includes components for automated sensor data collection, transport to a remote secure database repository, individualized subject model development, and subject state classification based on new sensor fusion methods hosted on a server (e.g., the UCLA DataServer repository). In some embodiments, a summary of the activity data is integrated with additional patient-specific information drawn from the electronic medical record of patients.

The system is amenable to the development of many sensor-based algorithms that are trained to recognize most real-world daily activity patterns, such as reaching with the upper extremities, eating, washing, exercising with equipment, standing up, walking at any speed, climbing stairs, and, with GPS or voice notation, walking in the community. In some embodiments, the network also integrates sensors into exercise equipment to record forces exerted by subjects. Moreover, in some embodiments, information from a broad range of sensors either worn on the person or embedded in the environment is integrated and processed. Consequently, the present invention allows for an extremely broad range of conditions to be studied at lower cost, the relative effectiveness of different therapies to be evaluated, and the home care of millions of patients to be improved.

In some embodiments, the PAMs monitor exercise compliance across complex assigned tasks and give patients immediate or delayed feedback via a computer, email, or phone call as soon as data is downloaded. This information enhances compliance and enables investigators and clinicians to progressively increase the demands of skills training, conditioning, and strengthening tasks without the need for patients to travel to a clinic or pay for their daily therapy. The ability of clinicians to monitor health-related activity with feedback also improves clinical effectiveness by promoting daily patient engagement, behavioral change, and more self-management.

In some embodiments, the PAM-based activity monitoring of the present invention provides information that will assist a health care provider regarding subject activity and the intensity of informal practice. Indeed, using the PAM-based activity monitoring of the present invention, pilot studies of new interventions develop more exact dose-response data to optimize the intensity of a therapy, prior to conducting an RCT.

In some embodiments, the PAM-based system adds rehabilitation-related data to physiological parameters. In some embodiments, these physiological parameters are obtained by telemonitoring via sensors for heart rate, blood pressure, and/or electrocardiogram. It is anticipated that data acquired from combinations of wireless wrist, ankle, and waist-fitted PAMs offers opportunities to design new strategies for pilot and Phase 1 to 3 trials, based on the availability of clinically meaningful daily monitoring and repeated outcome measures of the effects of pharmacologic and physical therapies.

In one aspect of the present invention, a system for monitoring patient activity comprises at least one measurement device and a server. The measurement device is configured to provide data related to a patient's physical activity, and the server is configured to make an inference regarding the physical activity based on data provided by the measurement device. In some embodiments, the inference is a determination of a type of physical activity.

In some embodiments, the measurement device is configured to provide the data related to the patient's physical activity from a location remote from the server. In some embodiments, the measurement device is configured to be worn by the patient or carried in the patient's pocket.

In some embodiments, the measurement device is configured to transmit the data related to the patient's physical activity via wireless communication. In some embodiments, the system comprises two or more measurement devices each configured to provide data related to the patient's physical activity. In some embodiments, the measurement device comprises a triaxial accelerometer, a microgyroscope, or a pressure sensor. In some embodiments, the measurement device is configured to automatically take repeated data samples.

In some embodiments, the server is configured to infer the probability of a patient being in an activity state based on the data provided by the measurement device. In some embodiments, the server is configured to make the inference based on a combination of data obtained from different measurement devices corresponding to different parts of the patient's body. In some embodiments, the data in the combination of data is based on samples being taken simultaneously by the different measurement devices. In some embodiments, the server is configured to apply Bayesian Sensor Fusion analysis in making the inference. In some embodiments, the server is configured to apply a naïve Bayer classifier model to infer the probability of a patient state vector given a feature vector. In some embodiments, the server is configured to use a Fourier transform in processing data provided by the measurement device in a time domain to extract frequency spectral components. In some embodiments, the server is configured to use a Fast Fourier transform. In some embodiments, the server is configured to use a fundamental frequency component and spectrum energy in making the inference. In some embodiments, the server is configured to make the inference by applying one or more motion recognition algorithms In some embodiments, the server is configured to make the inference by applying one or more state classification algorithms In some embodiments, the server is configured to archive and retrieve the data provided by the at least one measurement device and the inferences.

In another aspect of the present invention, a method of monitoring patient activity comprises a server receiving data related to a patient's physical activity, wherein the data is based on one more samples from at least one measurement device, and the server making an inference regarding the patient's physical activity based on the received data. In some embodiments, the inference is a determination of a type of physical activity.

In some embodiments, the server is located remotely from the measurement device. In some embodiments, the step of the server receiving the data is preceded by a step of the measurement device taking one or more samples of the patient's physical activity. In some embodiments, the measurement device is worn by the patient or carried in the patient's pocket when the one or more samples are taken. In some embodiments, the measurement device transmits the data via a wireless communication. In some embodiments, the measurement device comprises a triaxial accelerometer, a microgyroscope, or a pressure sensor. In some embodiments, the measurement device automatically takes repeated data samples.

In some embodiments, the server infers the probability of a patient being in an activity state based on the data provided by the measurement device. In some embodiments, the server makes the inference based on a combination of data obtained from different measurement devices corresponding to different parts of the patient's body. In some embodiments, the data in the combination of data is based on samples being taken simultaneously by the different measurement devices. In some embodiments, the server makes the inference by applying Bayesian Sensor Fusion analysis. In some embodiments, the server applies a naïve Bayer classifier model to infer the probability of a patient state vector given a feature vector. In some embodiments, the server uses a Fourier transform in processing data provided by the measurement device in a time domain to extract frequency spectral components. In some embodiments, the server uses a Fast Fourier transform. In some embodiments, the server makes uses a fundamental frequency component and spectrum energy in making the inference. In some embodiments, the server applies one or more motion recognition algorithms in making the inference. In some embodiments, the server applies one or more state classification algorithms in making the inference. In some embodiments, the method further comprises the server archiving the received data and the inferences for subsequent retrieval.

In yet another aspect of the present invention, a program storage device readable by a machine tangibly embodies a program of instructions executable by the machine to perform a method of monitoring patient activity. The method comprises making an inference regarding a patient's physical activity based on data related to the patient's physical activity, wherein the data is based on one more samples from at least one measurement device. In some embodiments, making an inference comprises determining a type of physical activity.

In some embodiments, the method further comprises inferring the probability of a patient being in an activity state based on the data provided by the measurement device. In some embodiments, the method further comprises making the inference based on a combination of data obtained from different measurement devices corresponding to different parts of the patient's body. In some embodiments, the data in the combination of data is based on samples that have been taken simultaneously by the different measurement devices. In some embodiments, the method further comprises applying Bayesian Sensor Fusion analysis in making the inference. In some embodiments, the method further comprises applying a naïve Bayer classifier model to infer the probability of a patient state vector given a feature vector. In some embodiments, the method further comprises using a Fourier transform in processing data provided by the measurement device in a time domain to extract frequency spectral components. In some embodiments, the method further comprises using a Fast Fourier transform in processing data In some embodiments, the method further comprises using a fundamental frequency component and spectrum energy in making the inference. In some embodiments, the method further comprises applying one or more motion recognition algorithms in making the inference. In some embodiments, the method further comprises applying one or more state classification algorithms in making the inference. In some embodiments, the method further comprises archiving the received data and the inferences for subsequent retrieval.

In yet another aspect of the present invention, a system for training a model for monitoring patient activity comprises a server configured to extract features from training data, cluster the extracted features into a discrete feature space, and perform a maximum likelihood estimation for the discrete feature space to construct a maximum likelihood model. In some embodiments, the server is configured to cluster the extracted features using Gaussian cluster discretization. In some embodiments, the server is further configured to correlate features with different states of activity for a patient.

In yet another aspect of the present invention, a method of training a model for monitoring patient activity comprises extracting features from training data, clustering the extracted features into a discrete feature space, and performing a maximum likelihood estimation for the discrete feature space to construct a maximum likelihood model. In some embodiments, clustering the extracted features comprises performing Gaussian cluster discretization. In some embodiments, the method further comprises the step of correlating features with different states of activity for a patient.

In yet another aspect of the present invention, program storage device readable by a machine tangibly embodies a program of instructions executable by the machine to perform a method of training a model for monitoring patient activity. The method comprises extracting features from training data, clustering the extracted features into a discrete feature space, and performing a maximum likelihood estimation for the discrete feature space to construct a maximum likelihood model. In some embodiments, clustering the extracted features comprises performing Gaussian cluster discretization. In some embodiments, the method further comprises the step of correlating features with different states of activity for a patient.

### BRIEF DESCRIPTION OF THE DRAWINGS

FIG. 1 illustrates a Bayesian diagram in accordance with some embodiments of the present invention.

FIG. 2 illustrates stages of a training process in accordance with some embodiments of the present invention.

FIG. 3 illustrates a Gaussian clustering for feature extraction in accordance with some embodiments of the present invention.

FIG. 4 illustrates a query pipeline in accordance with some embodiments of the present invention.

FIGS. 5A-B illustrate the acceleration with an upper extremity PAM in accordance with some embodiments of the present invention.

FIGS. 5C-D illustrate the direct and accurate subject state classification with the upper extremity PAM of FIGS. 5A-B in accordance with some embodiments of the present invention.

FIGS. 6A-B illustrate the acceleration with a lower extremity PAM in accordance with some embodiments of the present invention.

FIGS. 6C-D illustrate the actual and accurate subject state classification with the lower extremity PAM of FIGS. 6A-B in accordance with some embodiments of the present invention.

FIG. 7 illustrates the Z-axis acceleration with the lower extremity PAM of FIGS. 6A-B in accordance with some embodiments of the present invention.

FIGS. 8A-C illustrate a three-axis acceleration time series with a lower extremity PAM in accordance with some embodiments of the present invention.

FIGS. 8D-E illustrate the actual and accurate subject state classification for the lower extremity PAM of FIGS. 8A-C in accordance with some embodiments of the present invention.

FIG. 9 illustrates a PAM system operation in accordance with some embodiments of the present invention.

FIG. 10 illustrates a PAM system architecture in accordance with some embodiments of the present invention.

FIGS. 11A-B illustrate accurate and actual results of gait classification with bilateral distal leg sensors in accordance with some embodiments of the present invention.

FIGS. 12A-B illustrate the accurate and actual walking speeds corresponding to FIGS. 11A-B in accordance with some embodiments of the present invention.

FIGS. 13A-C illustrate the actual and accurate cadence measurements for each behavior in FIGS. 11A-12B as well as the ratio of right to left stride period in accordance with some embodiments of the present invention.

### DETAILED DESCRIPTION OF THE INVENTION

The following description is presented to enable one of ordinary skill in the art to make and use the invention and is provided in the context of a patent application and its requirements. Various modifications to the described embodiments will be readily apparent to those skilled in the art and the generic principles herein can be applied to other embodiments. Thus, the present invention is not intended to be limited to the embodiment shown but is to be accorded the widest scope consistent with the principles and features described herein.

The present invention can be provided as a computer program product that can include a machine-readable medium having stored thereon instructions that can be used to program a computer (or other electronic devices) to perform a process according to the present invention. The machine-readable medium can include, but is not limited to, floppy diskettes, optical disks, CD-ROMs, ROMs, RAMs, magnet or optical cards, or other type of media/machine-readable medium suitable for storing electronic instructions.

Furthermore, it is contemplated that any features from any embodiment can be combined with any features from any other embodiment. In this fashion, hybrid configurations of the disclosed embodiments are well within the scope of the present invention.

Various aspects of the disclosure can be described through the use of flowcharts. Often, a single instance of an aspect of the present disclosure can be shown. As is appreciated by those of ordinary skill in the art, however, the protocols, processes, and procedures described herein can be repeated continuously or as often as necessary to satisfy the needs described herein. Additionally, it is contemplated that method steps can be performed in a different order than the order illustrated in the figures, unless otherwise disclosed explicitly or implicitly.

The International Classification of Functioning, Disability, and Health (ICF) provides a framework for developing and selecting outcome measures for research in neurologic rehabilitation. In some embodiments, certain ICF categories are better operationalized by PAM data. The ICF starts with two structures, Functioning and Disability and Contextual Factors. Each structure includes two components. The component Activities and Participation describes domains of functioning from both an individual and societal perspective. In contrast to most other disability models, the ICF classifies Contextual Factors that may either facilitate or hinder functioning and influence disability. Contextual Factors include Environmental Factors in the physical, social, or attitudinal world and Personal Factors such as age, lifestyle, and coping. For example, in the ICF framework, several categories are related to locomotor coordination after stroke, under section “b” (Body functions): b710 (mobility of joint functions), b760 (control of voluntary movement functions), specifically b7602 (coordination of voluntary movements), and b770 (gait pattern functions). The classification of measures of walking under changing conditions is under section “d” (Activity or activity limitations), more specifically under the section “d4” (mobility; d450-d469, walking and moving). This means that the two constructs, locomotor coordination and mobility, are not at the same level of ICF classification and could be evaluated by different measures. These constructs do, however, interact closely. For example, a person who reaches a higher level of coordination may walk faster and obtain a better score on a functional mobility scale, but the gait pattern could be less safe and require more energy (e.g., extreme hip hiking), especially under differing demands. Walking speed tested in a laboratory environment may not reflect a desired improvement of coordination under more challenging physical or social Environmental Factors. The ICF also aims to show the dynamic interplay between impairments, activities, participation, health conditions, personal characteristics, and contextual factors. Participation in daily roles and most activities have been more closely related to QoL in disability models and assessed by questionnaires with ordinal scales, rather than considered within the level of impairment or functional performance. PAM-type movement data can quantify, in the example above, locomotor coordination and mobility both indoors and in the community to reflect and perhaps help integrate activities and participation within many of the ICF structures. In regard to SCI, Magasi et al. stated, “Given the importance of participation to people with disabilities, disability policy, rehabilitation research, and clinical practice, it is imperative that clinicians and researchers have access to outcome measures that accurately measure participation in ways that are both conceptually and psychometrically sound.” (Magasi S, Heinemann A, Whiteneck G. Participation Following Traumatic Spinal Cord Injury: An Evidence-Based Review for Research. J Spinal Cord Med 2008; 31:145-56). Sensor-based activity data has the ability to benefit studies of participation across diseases, as well as most directly reveal new data about activities wherever subjects go. The deployment of PAMs to construct clinical measures of mobility, balance, and upper extremity use in the home and community reduces questionnaire-based limitations in detecting activities and participation across ICF structures. Perceived problems of patients are directly assessed and the interactions between ICF structures and actual behavior adds to the understanding of barriers and facilitators of function.

Rehabilitation and health services researchers have developed valued generic QoL (e.g., Sickness Impact Profile, Medical Outcomes Study SF-36) and functional disability or burden-of-care ordinal scales (e.g., Functional Independence Measure, Frenchay Activities Scale, Modified Rankin Scale), as well as disease-targeted scales (e.g., Stroke Impact Scale, Quadriplegic Index of Function for SCI, Extended Disability Status Scale for MS). What patients can and cannot do that is important to them, as well as what they actually do in terms of activity and participation, may not be captured by these various tools. To do so has become so important as a public health and outcomes indicator that the NIH is sponsoring the Patient-Reported Outcomes Measurement Information System (PROMIS). This NIH Roadmap initiative will develop a computerized system for patients across diseases to identify symptoms and QoL outcomes most relevant to them (http://www.nihpromis.org). A related strategy is to create a quantitative scale, such as the Activity Measure for Post Acute Care, by pooling multiple items that assess a functional concept. With 101 functional activity items that relate to ICF activity subdomains, a mobility score, for example, would be based on the level of difficulty walking at home, outdoors, and arising from a chair, among others. The combination of a nonhierarchical measure like the ICF and a quantifiable scale like the AMPAC or Functional Independence Measure, after appropriate psychometric techniques such as Item Response Theory and Rasch analysis are applied, may produce better defined staging of the activities that can and cannot be performed. The potential for PAM sensors to recognize the quality and quantity of activity patterns in real-world environs could help validate and expand these efforts. Indeed, a potentially more optimal solution than creating larger and more complex ordinal scales may be to develop high quality instruments that quantify activities and participation during performance This data could be obtained in the community, including from those who are disenfranchised by their disability or their social and economic disadvantages.

Functional and QoL tools with hierarchical ordinal scaling are usually secondary outcomes in clinical trials for motor-related interventions. Primary outcomes have increasingly become timed measures of a particular activity or battery of activities performed in a laboratory setting or by a highly validated scale of impairment such as the Fugl-Meyer Motor Assessment. For the upper extremity, for example, interval measures such as the Nine Hole Peg Test and Wolf Motor Function Test (WMFT) capture some of the functional abilities required for upper extremity ADLs and Instrumental ADLs. These single or multi-item interval scales offer greater sensitivity to change especially over the course of a short intervention, but have inherent potential for measurement errors. In relation to the ICF model, these tests may not cover the capacity for the range of upper extremity movements that are important to patients. Also, the timed tasks cannot directly reveal the level of limb activity beyond a laboratory setting. They must be supplemented by other scales.

Occasionally, continuous measures are obtained from kinematic analyses to show speed and trajectories of reaching movements, although expensive equipment and expertise are required. Activity monitoring using an accelerometer on the arm reveals the amount of movement, but prior to the PAM activity-pattern recognition algorithms of the present invention, not the type or quality of purposeful arm activity. Commercial devices do not save data points frequently enough to capture details of movements and cannot classify functional from nonfunctional accelerations and decelerations. When combined with a structured interview about the amount of activity, such as the Motor Activity Log (MAL), these devices help validate the subject's perception of amount of use. On the other hand, the MAL has some inherent weaknesses found in all self-report measures, including recall and availability bias, subjectivity, demand characteristics, and experimenter bias. In some embodiments, the PAM sensors of the present invention eliminate these confounders because of their continuous activity pattern recognition in any environment, as well as their potential to measure the speed and quality of the trajectory of movements. Thus, direct PAM-based observation could become a gold standard for measuring purposeful upper extremity activities.

Commonly used mobility and balance tools include the timed up-and-go (TUG), Berg Balance Scale, walking speed for 6-10 m or a 50-foot walk, the distance walked in 2-6 min, and force plate-based center of balance measures. The short distance walking speed at a casual or fastest pace has been the most frequently used primary outcome measure in recent multicenter RCTs. Walking speed is also one of 3 components of the MS Functional Composite outcome measure. The Phase 3 trial of fampridine recently sought FDA approval for patients with MS based on finding a 25% increase in walking speed in responders. Approval was also sought based on a complementary significant increase in scores of these patients on the multiple sclerosis walking scale (MSW-12) that offers the patient's perspective on disability for ambulation. Of interest, most elderly persons and patients with stroke and MS can increase their walking speed for 6-15 m by 15%-25% over their casual speed when encouraged. More importantly, the subjective estimate by investigators of a decrease in disability or increase in participation in activities that depend on mobility may be moot when relying on the laboratory walking speed and a functional scale. Indeed, in one example, the clinic-based 10-m walking speed of patients after stroke did not predict walking velocity in a community setting that was artificially set up. In patients who walked at less than 0.8 m/s in the clinic test, gait velocity outdoors was overestimated. Laboratory walking speed and distance may not be an accurate measure of how fast and how far patients walk on paved streets, crossing intersections, on sloped aisles at theaters or in human traffic at a market. However, in some embodiments, the PAMs of the present invention are configured to measure maximum and minimum speeds at short and long intervals over the course of community activities, along with energy consumption.

Formal 3-D video-based motion analysis as a measurement tool is expensive and requires expertise to define kinetic, kinematic, and spatiotemporal aspects of the gait cycle. A major confounder for use of this data is that subjects take only about 6-8 steps, wear a lot of gear, and must hit a hidden force plate with at least one step, all of which may alter their natural walking characteristics compared to outside of the laboratory. The sophisticated gait lab serves many useful purposes for research, but cannot reflect how people walk under home and community conditions. Spatial and temporal gait parameters can also be collected using an electronic gait mat that has embedded pressure sensors and relies on a stop watch for speed (GAITRite, CIR Systems Inc.), but is only about 5 m in length. Thus, this test samples speed and limb symmetries that may change after an intervention, but would not necessarily be paralleled by changes in home and community mobility activities. In some embodiments, the PAM accelerometers of the present invention are configured to detect changes in real-world settings using activity recognition algorithms

Accelerometry has made some inroads for rehabilitation studies that could be extended, if inexpensive devices with activity recognition patterns were available. For example, motion-sensitive pedometers have counted the number of steps (if artifacts could be excluded), but not the quality of stepping or speed. Several types of biaxial and triaxial accelerometers have also been used, mostly in lab-based research. Most were developed to measure METS or activity counts (units of active mobility). The $500 RT3 (Stayhealthy, Inc.) represents this type when worn at the waist, but a study found that it had to be used in conjunction with a daily activity diary in persons with stroke or MS to measure daily physical activity. Of note for DAWN studies, activity studied for a week was more reproducible than data acquired for only 3 days. A triaxial piezoresistant accelerometer, fastened with an elastic belt over the L3 spinous process (DynaPort MiniMod, McRoberts) can define stance and swing times to detect asymmetries between the legs, as well as walking speeds that are >0.5 m/s. A $2500 fragile system of 5 wired biaxial accelerometers (e.g., IDEEA, Minisun) placed over the thigh, sole and sternum reveals not only spatiotemporal aspects of gait, but specific activities such as a walking versus stair climbing, but has limited accuracy in patients with walking speeds <0.4 m/s. The repeatability and reliability (ICC+0.9) of acceleration-based gait analysis has been very high for detecting cadence, speed, asymmetry in stance and swing times, and step length, along with irregularities associated with turns and changes in speed in healthy subjects and hemiparetic patients after stroke. The use of the present invention has reproduced data equivalent to the IDEEA and DynaPort using a PAM on each ankle or at the waist and has detected gait speeds as low as 0.25 m/s. A central problem for the deployment of commercial devices by rehabilitation researchers is that they all use proprietary data analysis systems created to detect very specific movements. In some embodiments, the data algorithms of the present invention will be accessible and can be continuously developed to meet the needs of researchers and clinicians.

Most embedded systems are designed for a single purpose, with a closed architecture. Medical monitors in particular are seldom designed for multiple user communities, typically being targeted either only at the patient or the medical professional. Either no data goes to the medical professional, or it is presented in only a limited number of ways. The PAM system of the present invention presents a new paradigm for personalized health care. It is an end-to-end modular system in which the patient, family members, nurses, physicians, and medical researchers can all access data via interfaces that can be specialized to their very different needs. Low cost and robust physical monitoring devices are paired with server systems enabling sophisticated and flexible analysis of data. A layered processing architecture enables high-priority events to be quickly flagged, and the data to be searched and processed to meet differing goals over time. A common look and feel coupled with built-in inference engines enables new monitoring devices to be added, expanding the scope of applications, while minimizing re-training in how to effectively use the system, thus providing patients and caregivers with unprecedented feedback concerning compliance with therapy, effectiveness of therapy, and providing quantitative records that enable both improved individual care regimes and low-cost studies across large populations.

The PAM signal processing and state classification system of the present invention provides a fundamental advance over conventional activity monitoring systems. The system architecture provides several distinguishing features. In some embodiments, the architecture permits automatic classification of diverse individual behaviors with a system that may be trained rapidly and accurately without expert administration. In some embodiments, the architecture is housed within a low cost, compact, low mass, waterproof device that can be worn on limbs or carried in a pocket. Advances in microelectronics, in low power design, and context-specific sensing provide long operating life using a rechargable battery. Advances in non-volatile memory afford a long term (e.g., 1 week) for data acquisition. In some embodiments, the architecture integrates data transport, archiving, processing, and data sharing with required capabilities to ensure privacy, providing data de-identification, and other services. In some embodiments, the architecture includes a sequence for signal processing, multiple sensor data fusion, and ultimately high-resolution subject state classification via a PAM DataServer system. In some embodiments, the architecture operates with multiple sensor axes at high sample rate ensuring sufficient time resolution for each of the many medical applications. In some embodiments, the architecture enables insertion of the PAM device into a standard computer USB port for automatic recognition and for uploading records to the remote PAM DataServer. In some embodiments, no data remains on the PAM. In some embodiments, the architecture utilizes data travel over the standard Secure Shell (SSH) protocol for devices and via Hypertext Transfer Protocol over Secure Socket Layer (HTTPS) protocols for web interfaces. In some embodiments, subject devices are provided with unique Secure Sockets Layer (SSL) (http://tools.ietf.org/html/rfc5246) keys that ensure authentication and secure data transport. In some embodiments, subject user registration processes provide individual keys.

In some embodiments, the PAM is based on or utilizes the MicroLEAP wearable motion sensing system that is commercially available. The triaxial accelerometer data allows detailed detection of subject activity states. In some embodiments, the PAM devices are extended to include rotation rate (microgyroscope) and pressure sensors, as when they are integrated into assistive devices such as the Smart Cane. PAM accelerometry data has been used by the inventors of the present invention to monitor athletes during track and field events and emergency response workers such as firemen as they manage the physical demands of a fire. Activity classifications, energy cost of individual activities, and quantity and quality of movements have been successfully measured over the course of this research. The statistical procedures discussed in detail below to identify activity patterns have been made more robust: (1) by having subjects perform the tasks to be monitored and then using this template to assist pattern recognition; and (2) by combining simultaneous sensor data from the arms and legs to assess whether one or more limbs are involved in performing synchronous versus non-synchronous, symmetric versus asymmetric or alternating tasks.

The fundamental advance of the present invention's PAM state classification system is the ability to retrieve and archive a subject's data on a remote server, while applying a variety of motion recognition, state classification and behavior learning algorithms that are configured to give this data clinical utility. Furthermore, in some embodiments, all of these systems automatically operate on the remote server and can be expanded or reconfigured at any time, thus permitting continuous advances by the DAWN team.

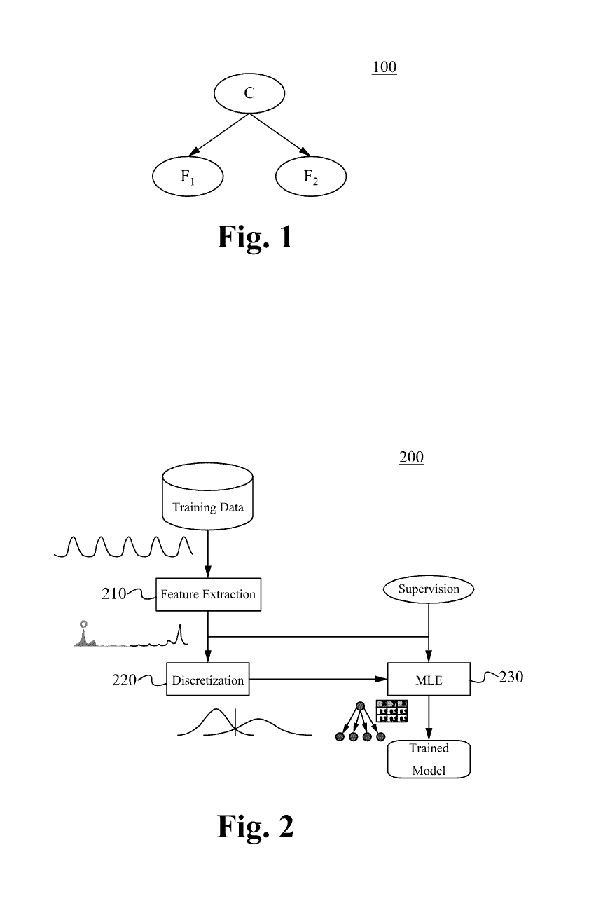

In some embodiments, the PAM classification method is based on Bayesian Sensor Fusion analysis principles. The Bayes approach establishes a relationship between the subject state, C, that is to be classified with sensor data features, F (specifically the evidence of C). This approach is based on the so-termed “naïve assumption” that the evidence or data features F are conditionally independent. Correlation between the different features providing the evidence of C is not included in this analysis. This method has been successful over a broad range of biomedical applications including those associated with motion analysis. FIG. 1 shows a simple Bayesian diagram that describes a causal relationship between the class C and the two sources of evidence F₁ and F₂. This Bayesian method allows computational efficiency and robust operation with respect to noise in data.

The “naïve” Bayes formulation can be stated as:

$$p ( C \vert F ) = \frac{p ( C ) {\prod }_{i = 1}^{n} p ( {F}_{i} \vert C )}{{\prod }_{i = 1}^{n} p ( {F}_{i} )} \qquad ( 1 )$$

The equation can also be understood as relating posterior probability to prior probability, likelihood, and evidence regarding inference and data Note, that the denominator of Equation 1 is completely described since the values of all Fᵢ are known.

The feature extraction step summarizes the time domain data from each sensor into a vector of sensor feature variables F=[F1, . . . , Fn] for the Bayes classification step. In some embodiments, for patient ambulation in steady state, sensor readings in the time domain are processed to extract frequency spectral components by Fourier transform. The fundamental (single dominant) frequency component and spectrum energy have been found to be important features for physical activity recognition. In addition, the Fast Fourier Transform (FFT) is attractive due to its efficient computation. For example, suppose for a time-series sensor data x(t), X(k)=FFT(x(t)) and N is the length of the FFT vector, then the evidence extracted from the fundamental frequency component and spectrum energy feature can be summarized as the following:

$$\begin{matrix}{f}_{amp} = {max}_{k} ‖ X ( k ) ‖ & ( 2 ) \\ {f}_{energy} = {\sum }_{k = 1}^{N / 2} X ( k ) * X ( k ) & ( 3 )\end{matrix}$$

In some embodiments, the PAM Subject State Classifier architecture includes a library of features that have been applied successfully in the past and are available for evaluation for any new application. Furthermore, in some embodiments, the classifier architecture is inherently a modular design, enabling the use of other feature extraction procedures without changes to the other parts of the system. The output of the feature extraction step is a feature vector, F=[F1, . . . , Fn], extracted from the sensor data.

In some embodiments, the classification system applies the naïve Bayes classifier model described above in Equation 1 to infer the probability of the patient state vector C given the feature vector F from the feature extraction algorithm. As a result of this step, the system infers the probabilities for the patient being in one of the states C given the sensor feature vector extracted in the previous step. Note that the numerator of the classifier in Equation 1 essentially represents a product between the prior and the model that probabilistically relates the feature vectors to different patient states.

The Bayesian classifier described above relies on a probabilistic model of features, classes and their relation. In some embodiments, this model is trained within the PAM Subject State Classification system. Here, the system works on samples of sensor data that correspond to each of the states that need to be classified. The system needs to know which sensor data sample corresponds to which state. In machine learning jargon, this training of data is supervised to create a model, mapping input feature vectors to one of the several output classes by reference to several input-output examples of the classifier. In some embodiments, training data is applied to: (1) determine the number of discrete attributes (Equations 4 and 5) required in each feature variable in the naïve Bayes classifier; and (2) determine the likelihood of the feature variables (Equation 6) in the supervised learning. FIG. 2 illustrates stages of a training process 200 in accordance with some embodiments of the present invention. In some embodiments, the training process includes three stages: Feature Extraction 210, Gaussian Cluster Discretization 220, and Maximum Likelihood Estimation (MLE) 230.

The Feature Extraction step 210 was described above. At the step 220, the features extracted from the training data are clustered into a discrete feature space to learn the parameters of the Bayes classifier in linear time. Discretization also improves the performance of a naïve Bayes classifier significantly. A priori knowledge of the feature distribution in the training data set is unknown (hence, not assumed). The Gaussian (normal) probability distribution is thus assumed in order to maximize the information entropy and allow model-free clustering without requiring an explicit underlying domain model. Moreover, Gaussian clustering is computationally inexpensive and is based on a well-understood statistical model. In some embodiments, the Gaussian clustering includes two steps. First, the mean (μ) and standard deviation (σ) values representing the Gaussian clusters of the data points in the various supervised training types are extracted for each feature of each sensor. Second, the Gaussian clusters are then used to transform the continuous data space of each feature into the discrete states. The continuous data space of each feature F can be described by C Gaussian probability distributions (G(F)={G_{C}(F)}, ∀ ε {1, . . . , C}), corresponding to each supervised training type, c. A set of discrete intervals I is formed from the old continuous G(F) with each interval iⱼ ε(uⱼ, vⱼ], where uⱼ and vⱼ are the lower and upper boundary values, respectively. An interval boundary between vⱼ and uⱼ₊₁ is formed at vⱼ only when both conditions are met as shown in Equations 4 and 5.

vⱼ>G_{C}(F; μ_{C}+δ, α_{C}) (4)

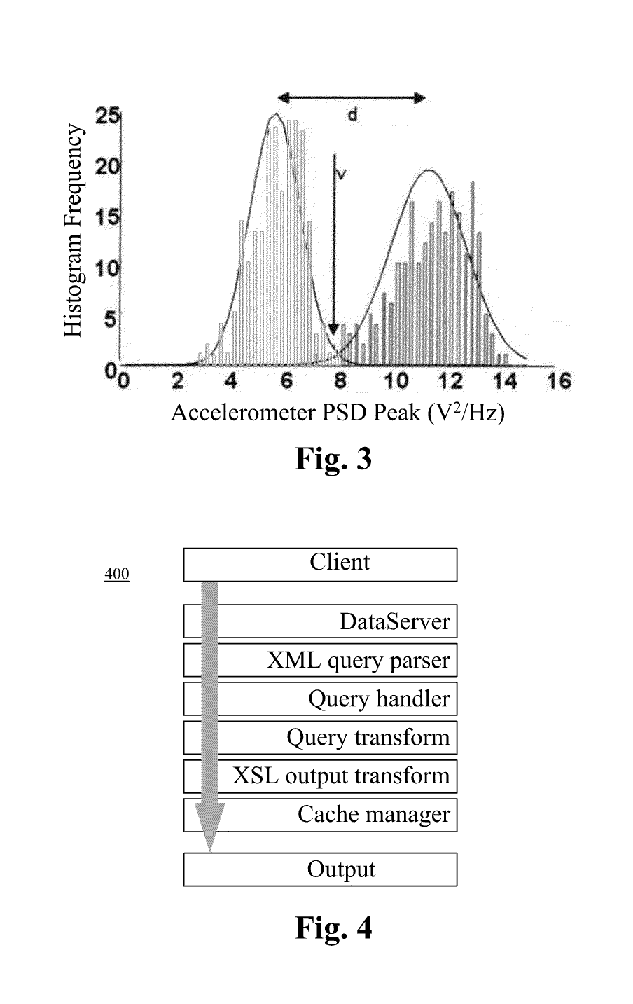

vⱼ<∀_{c′=c+1}^{C}(i_{C′}(F; μ_{C′}+δ, α_{C′}) (5)

where μ_{c}<μ_{c′} from the pre-sorting of the Gaussian distributions by their mean values, and δ is a threshold number based on σ. The feature values f from the original continuous space are mapped to the discrete ij and all relevant probabilities are estimated with respect to ij. FIG. 3 shows an example of Gaussian clustering for features extracted from the accelerometer signal that correspond to 2 distinct classes. The arrow with a ‘v’ points to the boundary that separates the two classes.

Once the data space of the input feature vector is discretized, the maximum likelihood model can be constructed at step 230. This model is then used during the real-time classification at the server. In some embodiments, the conditional likelihood term in the Bayes classifier (Equation 1) is now trained through supervised learning by assigning the class labels during the training, which associates the input feature vectors with a given class in the Bayes classifier:

$$p ( {F}_{i} = {f}_{i} \vert C = c ) = \frac{count ( {F}_{i} = {f}_{i} ⋀ C = c )}{count ( C = c )} \qquad ( 6 )$$

As a result of the training step, the naïve Bayes model is constructed to correlate sensor features with different subject states of activity.

PAM architecture is inherently extensible to include sensor data fusion from multiple devices. The PAM classifier system, for example, has been tested for multisensor fusion in gait analysis with the objective of quantitatively estimating limp amplitude and for the Smart Cane. In this case, the PAM classifier system was combined with a decision support system that identifies the sensor set that provides the greatest contribution to increasing the certainty of inference regarding subject state. PAM accelerometers have been applied to the wrists and ankles, thighs, upper arms, and low back to test the best approaches for the classification of activities and to assess factors such as movement speed and joint moments, thereby allowing calculation of the ratio of involved versus uninvolved arm use, identification of nontask-related movements, and common task-related functional movements that may involve several limbs and the trunk. Additionally, limb data has been merged with that from rehabilitative exercise equipment where PAM devices measure force and torque. It is contemplated that further support research will add and configure sensor systems along a path that establishes reliability and validity of activity classifications. The low cost and compact geometry of a PAM device makes this feasible.

In some embodiments, data delivery to researchers and users relies on the DataServer. In 2004, the DataServer project was formally released as on open source project, and has been adopted in part by the Open Source Health Records Exchange project (OpenHRE.org) as a bridge to clinical data repositories through its caching and de-identification features. In some embodiments the present invention integrates PAM data into the medical record via DataServer, applying more complex server-side processing and rule analysis to provide feedback (e.g., updating sampling frequency based on a new lab value and increased patient activity). Data analysis and visualization tools integrated with DataServer are also available. Embedded within DataServer are security and de-identification protocols, along with automated logging/auditing to facilitate the use of collected data towards the development of research repositories and databases. DataServer thus provides a portal for the real-time integrated data aggregation and retrieval/querying of PAM data, alongside additional distributed information sources (e.g., clinical and research databases). This framework serves to realize a PAM database as a resource for a broader community of researchers.

FIG. 4 illustrates a basic query pipeline 400 for the DataServer in accordance with some embodiments of the present invention. At the client, an XML query request is sent by the application to a secure web site (e.g., HTTPS). The web server passes the XML query request to the DataServer. The XML query request is then parsed. The parser deconstructs the request into individual queries. Each query is passed to the query handler factory, determining the appropriate response based on the targeted data source. A query handler generates the corresponding low-level database query for the XML-encoded query (e.g., XSL stylesheet transform into SQL). The new query is sent to the appropriate data source to retrieve information. The returned results are translated into XML. XSL is utilized to further transform the results into a target representation. The results of the XSL transforms are cached and passed back to the DataServer, which in turn sends the results back to the client application.

In summary, sensor data acquisition and processing has evolved to the point that this technology can be applied to medical rehabilitation research. Some examples follow. A range of applications are contemplated for the development and utilization of PAMs. These applications include: (1) statistical studies relevant to the various components of reliability, validity, and responsiveness of movement activities within the context of existing measurement tools and across research sites, levels of impairment and disability, diseases, age, gender, ethnicity, and socioeconomic strata; (2) development of sensor components and their deployment to obtain objective interval and ratio outcome measures for movement-related rehabilitation outcomes, particularly in natural environments; (3) objective, continuous measures of compliance with an exercise prescription during clinical trials or care; (4) assessment of levels of exertion and quality of movements; (5) design or use PAM sensor data and activity algorithms to meet the opportunities and needs for pilot studies and RCTs as ecologically sound measures of activity; (6) find a single or a composite group of specific sensor outcome measurements of efficacy that can be transferred into effectiveness outcomes, by targeting a broad target population in real-world settings (in some embodiments, by integrating PAMs into RCTs); and (7) define strategies for iterative collaboration between engineers, clinicians, and scientists in early stage technology development, with the goal to accelerate the production and use of high quality, clinically-relevant solutions for movement rehabilitation.

PAMs and activity pattern recognition will likely find uses across the spectrum of neurorehabilitation research and clinical practice. As the technology evolves in directions recommended by its users, additions will be made to the PAM toolbox.

In some embodiments, the present invention is used in studies of intermittent movements, examples of which are discussed below.

First, the amount of daily physical practice by inpatients during so-called “intensive rehabilitation” has been remarkably low when formally studied by observation. The present invention has been used to acquire reliability data for the quantity and types of movements made by patients over the course of inpatient care for stroke, SCI, and critical illness polyneuropathies and myopathies. PAM signals are processed for activity patterns (see below) without knowledge of what activities were performed, then compared to videotaped segments of grooming, eating, standing up, exercising with elastic bands, pedaling, walking, etc. Of note, when the number of repetitions and types of activities are provided as feedback to patients and therapists on a daily basis, patients practice from 30%-125% more in mobility and upper extremity tasks during and in between formal therapy sessions. In some embodiments, this reinforcement strategy is tested across sites in the course of gathering reliability and validity data for each activity.

Second, sleep apnea and abnormal movements during sleep are very common after stroke and brain injury and with aging. These pathologies usually cannot be identified outside of a sleep laboratory. In some embodiments, PAMs are used during laboratory sleep studies to gather patterns of arm and leg movements that can then be detected in patients at home by their PAMs. The signals can be integrated with chest wall movements and heart rate. In some embodiments, the same approach is used to test for the number and duration of spasms in patients with SCI or dystonic movements.

Third, in some embodiments, the present invention is used to compare inpatient EEG and video monitoring of seizures to integrated arm and leg accelerometry signals. In some embodiments, a system is developed to quantify attacks and warn families about daytime and nocturnal partial complex, focal, and generalized seizures. In some embodiments, off-the-shelf electronics via a Bluetooth connection are used so that the wireless device can set off an alarm, dial an emergency number on a cell phone, or turn on an infrared camera to record an overnight event.

Fourth, QoL assessments of physical functioning, fatigue, pain and other perceptions that impact people with MS have challenged trialists. To identify solutions, the types of activities and quantity and quality of mobility and affected upper extremity actions performed over the course of a week are examined in patients disabled by MS who can still walk. In some embodiments, with this baseline information, a telemedicine-based trial of exercise, using PAMs to monitor compliance, assess the effects of feedback about performance and measures changes in home and community activities.

Fifth, the proportion of patients with functional independence after stroke declines annually for up to 5 years, and these effects are greatest for those with Medicaid or no health insurance. In some embodiments, community monitoring with PAMs is used to reveal actual indoor and outdoor barriers and levels of activity and participation beyond what subjects describe, and improve healthcare providers' ability to coach patients in ways to achieve national guideline recommendations for exercise and activity to reduce the risk of recurrent stroke, as well as enhance functional gains. In some embodiments, the PAM is used to investigate cultural barriers to the use of monitoring technology, and ways to overcome such barriers.

Sixth, in some embodiments, the present invention is used to monitor and characterize the movements of patients who are in a minimally conscious state, examining responses to stimuli and circadian rhythms

Seventh, a haptic feedback system that transmits ground forces to the mid thigh for lower-limb prostheses has been developed. In some embodiments, the PAMs serve as activity monitoring and outcome measures of balance and gait for the military servicemen who are fitted. In some embodiments, this feedback system and PAM assessment are extended to patients with severe sensory neuropathies who complain of imbalance and difficulty walking.

Eighth, a PAM monitoring system has been developed to enhance compliance with health-promoting behaviors, including Theraband-based resistance exercises, for patients with obesity. In some embodiments, outpatient PAM data is placed into a patient's electronic medical record, showing a summary of the types and quantity of activity for a week at a time. One aim is to provide education about health maintenance and risk factor reduction for diabetes, hypertension, obesity, and coronary artery and cerebrovascular disease. These studies have led to data input and analysis systems and interfaces for feedback to doctors and patients that will be shared with PAM users.

Ninth, in some embodiments, PAMs are incorporated into the monitoring of the activities and safety of their patients with brain and spinal cord injuries who are placed in accessible apartments on a campus. In some embodiments, PAMs are used by wheelchair users who are at risk for shoulder pain in order to assess arm movements during daily wheelchair use.

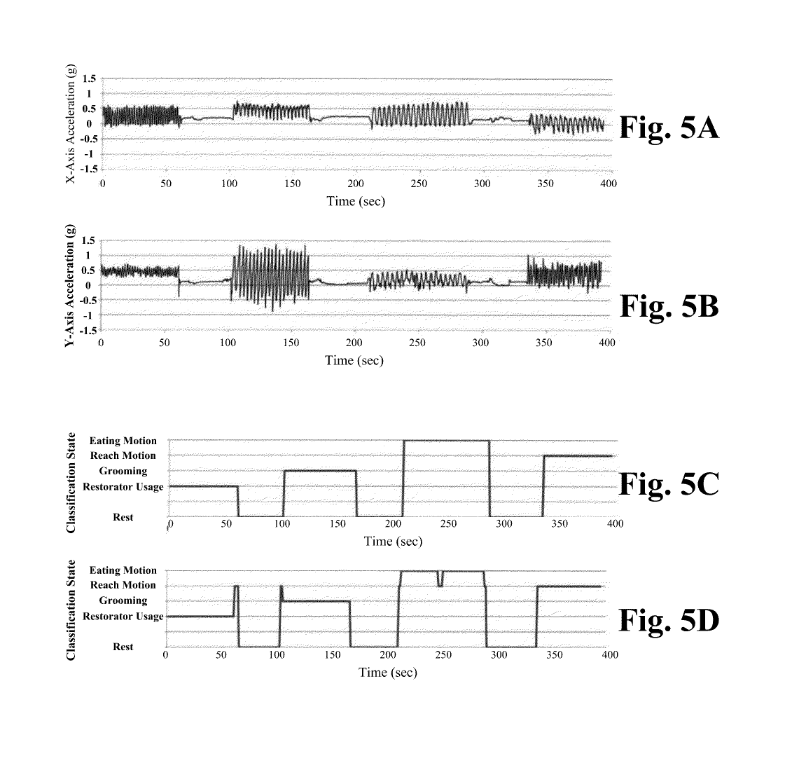

The above studies have had healthy control subjects and disabled persons perform various activities to test and train the Bayesian analytic approach already discussed. The following single-subject data was correlated with simultaneous kinematic and video recordings. FIGS. 5A-D show both a direct and then accurate subject state classification for arm movements. A healthy subject performed restorator cycling, hair grooming, eating from a plate (hand to mouth), and reaching for a cup movements (left to right columns). FIG. 5A shows X-Axis acceleration for a right wrist mounted PAM device (where positive X-Axis acceleration is oriented towards the hand), and FIG. 5B shows Y-Axis acceleration in the plane of the ulna and radius (where positive Y-Axis acceleration is oriented in the direction from ulna to radius). FIG. 5C displays the actual motion state for each episode over time. The vertical line for this and subsequent figures reaches the classified state and the horizontal line shows the reproducibility of each individual movement cycle for that state. FIG. 5D shows the accuracy of automatic classification of subject state using the Sensor Fusion State Classification system of the present invention.

Even without a hand sensor to detect grasp and release during reaching for an item, the algorithm uses the initial plane of movement, a decelerating stop when the object is reached, transport of the object toward the final destination, and another stop. In some embodiments, the smoothness of each directional movement is detected as well.

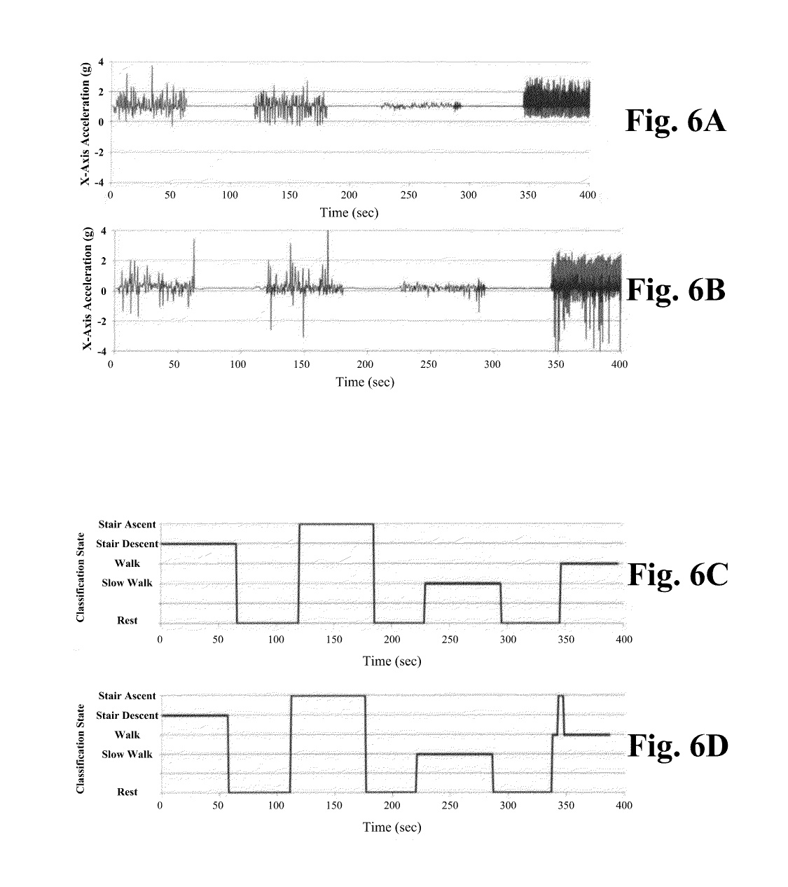

The same methods have been applied to classifier training of stair descent, ascent, slow walk and faster walk, as shown in FIGS. 6A-D from left to right columns. FIG. 6A shows Y-Axis acceleration for a right tibia-mounted PAM device (where positive Y-Axis acceleration is oriented towards the knee), and FIG. 6B shows Z-Axis acceleration (where positive Z-Axis acceleration is oriented in the forward direction). FIG. 6C displays the actual motion state for each episode over time. FIG. 6D shows the highly accurate results of automatic classification of subject state using the Sensor Fusion State Classification system of the present invention.

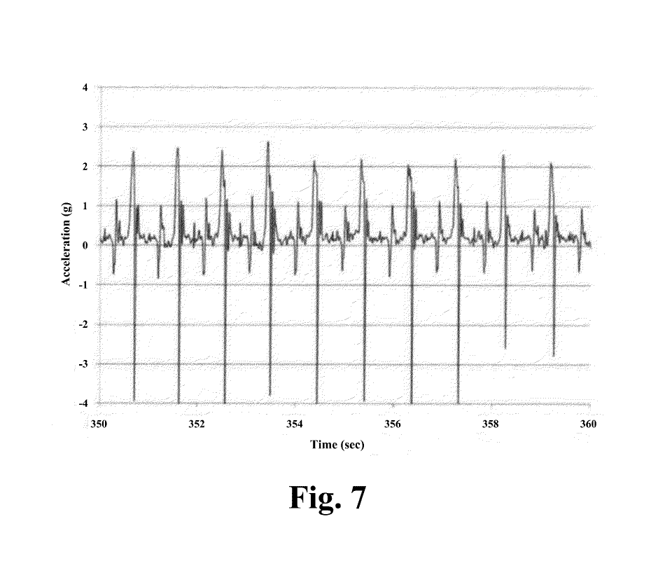

FIG. 7 shows a detailed short time series from the fourth column of walking in FIGS. 6A-D. The initial 1 g acceleration of each step cycle occurs after heel-off with toe-off, followed by the small deceleration with vertical lift of the foot. The buildup to the >2 g acceleration is the swing phase, followed by a large decelerating heel strike, and followed by the foot flat phase. These patterns are quite similar to the patterns of other, more expensive devices.

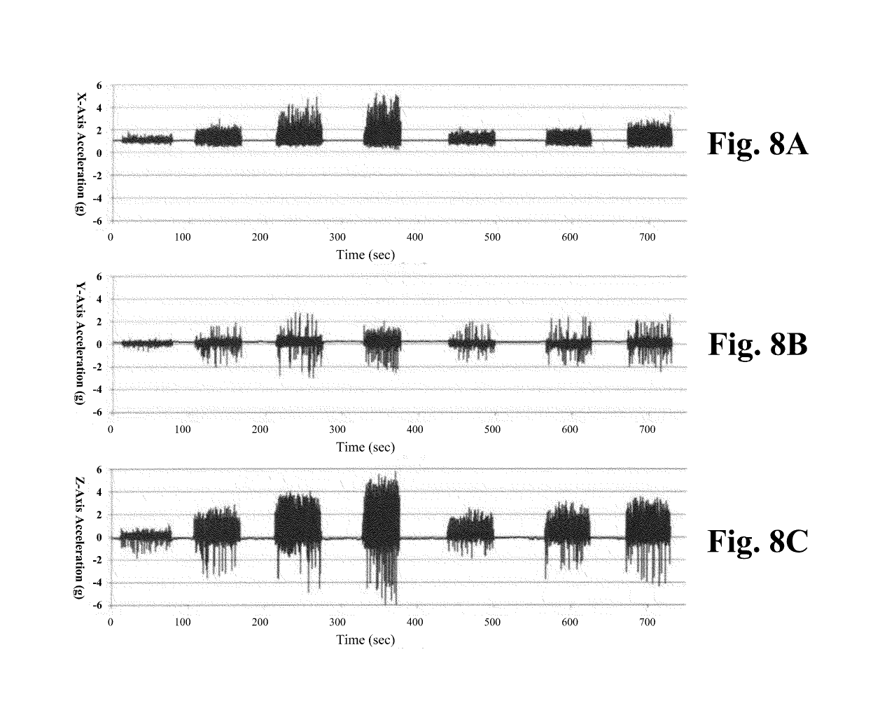

FIGS. 8A-C show a three-axis (X-Axis, Y-Axis, and Z-Axis) acceleration time series for a subject walking at 0.45, 0.9, 1.35, and 1.80 m/s. FIG. 8D shows the actual speed. Results in FIG. 8E show the highly accurate automatic classification of these speeds using the present invention. The walking speed for this subject were directly measured in a gait lab. The accelerometry data was then used for system training. This trained model was subsequently applied to unknown data to acquire the classification results shown. This method requires system training, but does not require other data for that individual, such as leg length or a stride length calculation. Asymmetries of the legs during walking are detected and spatiotemporal data is obtained using bilateral distal leg sensors. In some embodiments, adding leg length and other data further reduces the amount of system training.

Additionally, in some embodiments, a single thigh-mounted PAM device is used to classify sit-to-stand-to-sit events. Using the present invention, kinematic results were captured in a lab and the times at which the subject departed the seated posture, was in transition, and finally stood fully upright were noted. The sensor fusion system provided an accurate classification of each state. In some embodiments, over the course of a day or week, the patterns of activity or walking speeds are calculated automatically and visualized as a pie chart of the percent time spent in each state.

The present invention has potential clinical research applications. Based on the successful activity classifications, researchers will test PAMs for a wide variety of purposes. The system of the present invention enables reliability, validity, and sensitivity studies to be part of such research at low cost. Some examples are provided below.

In some embodiments, the present invention is used to optimize the reproducibility of a rehabilitation intervention to train specific skills by monitoring the quantity and quality of movements meant to be practiced by subjects under the guidance of therapists across trial sites.

In some embodiments, the present invention is used to monitor the pre-intervention activity of subjects in a clinical trial that may be most relevant to the intervention and to the primary outcome measurement, then stratify subjects prior to randomization based on high and low initial levels of activity.

In some embodiments, the present invention is used to capture the trajectory of gains or declines in the quantity and quality of specified activities using sensors. These measurements over time would enable investigators in their pilot studies to push an intervention until a plateau in gains is reached, rather than stopping the intervention after a pre-determined number of sessions or elapsed time. Thus, dose-response curves for an intervention and for combining interventions could be established, as they are in Phase 2 drug trials.

In some embodiments, the present invention is used to more closely monitor complex practice paradigms to learn which components of an intervention are most important. For example, are selective strengthening exercises less likely to improve outcomes than strengthening exercises that accompany functional movements in patients who have impaired upper extremity motor control?

In some embodiments, the present invention is used to examine the sustainability of the effects of interventions by using PAMs to inexpensively enable longitudinal studies. In some embodiments, “refresher measurements” of activity are performed at any time after the intervention without necessarily bringing subjects back to a laboratory.

In some embodiments, the present invention is used to gather longitudinal data from subjects of high interest, such as elderly persons or those with MS, to evaluate changes in their activities in relation to falls and injuries. In some embodiments, investigators record important events such as near falls or detect changes in walking speed and activity after a fall (e.g., as a consequence of fear of falling) with an optimal configuration of PAMs.

In some embodiments, the present invention is used to develop new measures for functional changes of the arm or leg after therapies for spasticity, such as botulinum toxin, which so far has not had much effect on daily mobility or purposeful arm activities.

In some embodiments, the present invention is used to refine what constitutes clinically useful functional walking speeds and distances necessary for home and community activities and participation. In some embodiments, norms are developed across diseases. These exist for stroke, but even here, PAMs permit the study of a large sample size with objective recordings of walking in varied environments.

In some embodiments, PAM data is used to get baseline and post-intervention data about how particular types of pain actually limit activities that are important to subjects.

In some embodiments, the present invention is used to improve testing for brain-behavioral relationships in neuroplasticity studies. In some embodiments, the cerebral activation paradigm during fMRI testing captures components of motor control for the foot or hand that are necessary to produce the movement skill being trained. In some embodiments, sensor behavioral recordings measure how often and how well those movements are made by subjects during their daily activities.

In some embodiments, the present invention is used to develop new scales of disability and activity with PAM data or augment existing scales and questionnaires to meet the specific demands of a trial.

In some embodiments, the present invention is used to enhance the application and testing of telerehabilitation protocols across diseases. Integrating PAMs into a telerehab protocol to monitor exercise, provide feedback, and to obtain activity-related outcomes serves patients who are remote from therapy centers or unable to travel.

A concern in rehabilitation is that evidence-based practices are not readily adopted by community therapists. Therapists and physicians have to grow comfortable and skilled in providing specific new exercise or task-oriented therapies. Clinicians may be more likely to adopt new evidence-based techniques if the same intervention, system for feedback, or outcome measures can be applied across a spectrum of pathologies. The PAMs may thus help enable the transfer of training paradigms and measures from the clinic into the community.

Animal studies suggest that exercise can improve aspects of cognition possibly by augmenting hippocampal neurogenesis and activating genes and molecular cascades for memory and learning. The subsequent behavioral changes in models of disease and aging vary in type and degree, but include improved motor speed and learning, cognitive processing speed, and auditory and visual attention. Such studies could test the hypothesis that skills learning and cognition can be enhanced by exercise and fitness training. Indeed, a trial of exercise was proposed by researchers from an Alzheimer's clinical consortium at the Apr. 21-22, 2009 NIH Blueprint workshop entitled “Harnessing neuroplasticity for human applications.” The fidelity of the exercise program is critical to any study of patients with mild dementia. In consideration of a possible home-based trial, the inventors of the present invention purchased a $25 commercial restorator and put pressure sensors on the pedals that communicate with a PAM on each wrist or ankle. This combination measures the force exerted by each limb and the number of repetitions per minute (RPMs), along with the duration of pedaling with the arms or legs. An inexpensive heart rate monitor was also attached to the chest and integrated with the data. This approach enables a trial to obtain a daily measure of compliance, gives subjects feedback about results, and lets a therapist progress the resistance or RPMs based on heart rate parameters and increasing aerobic gains while the subject is at home. The total cost for all equipment would be less than $150 for each subject in training (and reusable for later subjects). Thus, the trial could be managed by phone and email with only occasional personal evaluations, making an RCT financially more feasible and scientifically solid.

The PAM technology of the present invention enables clinicians who practice outside of academic centers to participate in community-based trials. In addition, the inclusion of their patients enhances external generalizability of a trial intervention to improve the health of community-dwelling patients. For example, inventors of the present invention recently completed a multi-center RCT that involved 20 inpatient stroke rehabilitation sites in 9 countries to test the feasibility of having neurorehabilitation clinicians become involved in clinical research using simple protocols (Walking Study for Stroke Rehabilitation—SIRROWS—clinical/trials.gov identifier NCT00428480). The study entered almost 200 subjects within 16 months. It showed that feedback about walking speed (stopwatch timed 10-m walk) during inpatient care leads to an increase in walking speed at discharge for those who received feedback (0.91 m/s vs 0.70 m/s) compared to those who were not informed about their daily gait speed. Length of stay was also significantly reduced. PAMs widen the potential for multi-site and international studies such as SIRROWS. They offer inexpensive compliance and outcome measurements with minimal overhead and training that can be deployed across languages and cultures. With PAMs, more sophisticated data collection than only walking speed also becomes feasible. For example, the concept of family-mediated therapy after stroke, which often happens with minimal if any professional support, could be tested using feedback about performance and progression of exercises that are relevant to the needs of patients in the community. In one scenario, patients and caregivers might take full responsibility for specific therapies to train balance, strength, fitness or a small set of skilled movements using feedback from therapists based on PAM data.

The present invention also has applicability in large animal research. PAMs have been employed on the hindlimbs of monkeys before and after reimplantation of lumbar nerve roots into the spinal cord following experimental avulsion lesions. This allows monitoring of limb use and activity during cage behavior, which may then replace complex video techniques. The system is capable or being extended to include devices for basic researchers who work with large animals.

Both our healthy control and disabled test subjects have been willing to wear the PAMs on ankles and wrists for a week without discomfort and replace them properly after taking them off over night. In some embodiments, devices are set within a 2×3 inch elastic pouch with velcro straps and slip under a pants leg, sock or shirt sleeve, so it is not anticipated that the great majority will resist using them, especially if the data is of value to subjects.

With the rapid expansion of applications for such systems, it is anticipated that new requirements for methods may be needed that support the classification of complex activities, classification that includes measurement in the presence of new noise and interfering signals, and support for tracking novel behaviors, as well as to assess changes in the same goal-directed movements as their quality improves over time. At least a few approaches will be taken to address these challenges as needed.

First, in some embodiments, extensions to the Bayes sensor fusion classifier are introduced. New classification methods include, but are not limited to, techniques such as the Boost Classifier and the Conditional Random Fields approach. Further, in some embodiments, the present invention utilizes methods that exploit information regarding the probability distributions describing sequences of events that occur in subject behaviors.

Second, the demand for in-field monitoring and validation of complex activities is anticipated. The PAM architecture supports incorporation of low cost video data sources. In some embodiments, the imaging platform employs the new low cost Atom processor platform that can be placed in a home or incorporated into assistive devices, such as the Smart Cane and wheelchairs. In some embodiments, video is initiated by a motion detector. Recordings will permit collaborators to access remote image data from subjects to evaluate specific behaviors and simultaneously capture sensor data for reliability and validity studies.

In addition, in some embodiments, a standard procedure to test for changes in the quality of movements of interest is built into pilot studies involving an intervention. For example, in some embodiments, the amount and quality of reaching movements is the goal of a rehabilitation strategy. To best monitor this change and to maximize pattern recognition by the algorithms, in some embodiments, subjects are asked to perform the WMFT before, at midpoint and immediately after a set of treatments while wearing the PAMs on a wrist or, if more detailed information about quality of movements is sought, on the wrist and mid upper arm. This updated data, obtained during a standardized series of timed movements, will serve both as an outcome measure across studies of collaborators and perhaps improve the pattern recognition of the sensor identification programs when subjects are outside of the laboratory.

In some embodiments, the PAM system readily fosters further technological development through a unique architecture that is based on server-hosted, signal processing in an open and conveniently configurable system. In some embodiments, the present invention comprises a Wireless Health Signal Processing toolkit that includes all of the system training, state classification, and data access and display tools necessary for high throughput processing of experimental data. In some embodiments, the toolkit also includes methods for precise performance measurement statistics. The PAM system will continue to advance in performance with the addition of classification methods that offer performance advantages over Bayesian methods. Further upgrades and advances will be introduced while maintaining compatibility with prior work. Finally, the didactic value of the toolkit is being continuously advanced.

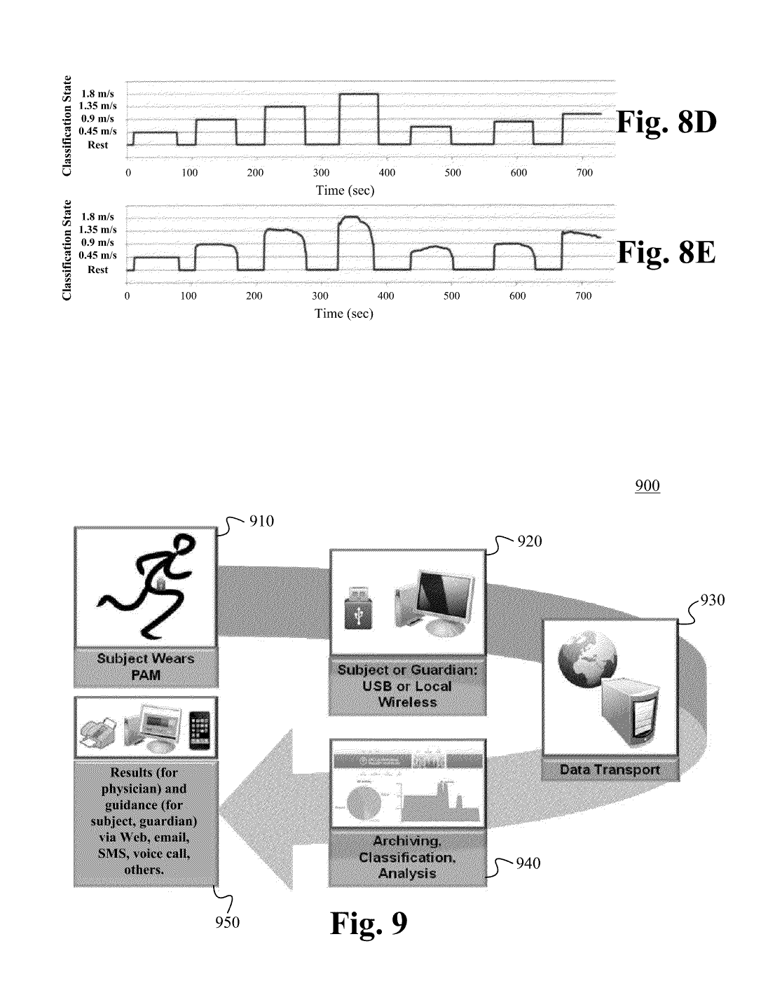

FIG. 9 illustrates a PAM system operation 900 in accordance with some embodiments of the present invention. In some embodiments, the PAM system comprises one or more measurement devices, preferably wearable by the subject. As seen in FIG. 9, the measurement device is worn or carried by the subject over a required monitoring period at 910. In some embodiments, the subject or guardian uses communication means that are provided on the measurement device or on optional additional devices in the environment, or both, in order to communicate with a server at 920. In this respect, the measurement device is communicatively coupled to the server. In some embodiments, the measurement device is periodically connected to a computer, such as by using a USB port or local wireless communication. In some embodiments, a portable computing device containing the PAM data, such as a cell phone, is brought by the subject or guardian into a physician's office, where the data is then transferred to the physician's computer. The computer can then be used to transmit the data to the server. In some embodiments, coupling to the server is achieved by means of standard secure internet protocols. Data from the one or more measurement devices is transmitted to the server via data transport at 930. In some embodiments, once the measurement device is plugged into a device, such as the physician's computer, configured to transmit the data to the remote secure server, the software application system recognizes the device and uploads all of the new data to the remote secure data server. In some embodiments, connection of the PAM device to a certain computer results in the automatic recognition of the PAM device and uploading of all records to the remote server. At the server, the data is processed, stored in a personalized database, and is automatically classified to identify subject state history by a variety of algorithms at 940. The server hosts a secure database in which the data from the measurement device is archived. The server also hosts algorithms for searching and interpreting the data, and tools for rendering the results in an understandable form for multiple user communities, thereby enabling the server to classify and analyze the data obtained by the measurement device. Finally, at 950, the processed data is available to the subject, guardian or doctor via a variety of different media, such as web access, SMS, fax, and others.

In some embodiments, the measurement devices comprise any one or combination of multiple accelerometers, sensor interfaces, a microprocessor, flash memory storage, battery, and a USB and/or Bluetooth radio port for downloading data and accepting new programming. In some embodiments, the measurement devices include cellular telephones or smart phones (preferably 3G or its successor), equipped with built-in sensors and localization devices such as GPS and/or communicating with other sensors via short range radios such as Bluetooth.

Patients, guardians, and physicians are able to interact with the system at 950 in order to obtain results and guidance. In some embodiments, this interaction includes, but is not limited to, web site communication, e-mail communication, SMS communication, voice call communication via the phone, entering voice inputs, and receiving visual and audio feedback. In some embodiments, the phone communicates with a computer controlling a television screen/home entertainment system to provide visual and/or audio feedback on performance of exercises or other physical tasks.

Complete systems including the server-side processing have been tested, and the Bayes classifier has shown to be reliable. Multiple alternative classifiers are being investigated using experimental data from the PAM system to increase robustness and are contemplated to be within the scope of the present invention. In some embodiments, data schema and search algorithms are used to enable rapid search of massive electronic records.

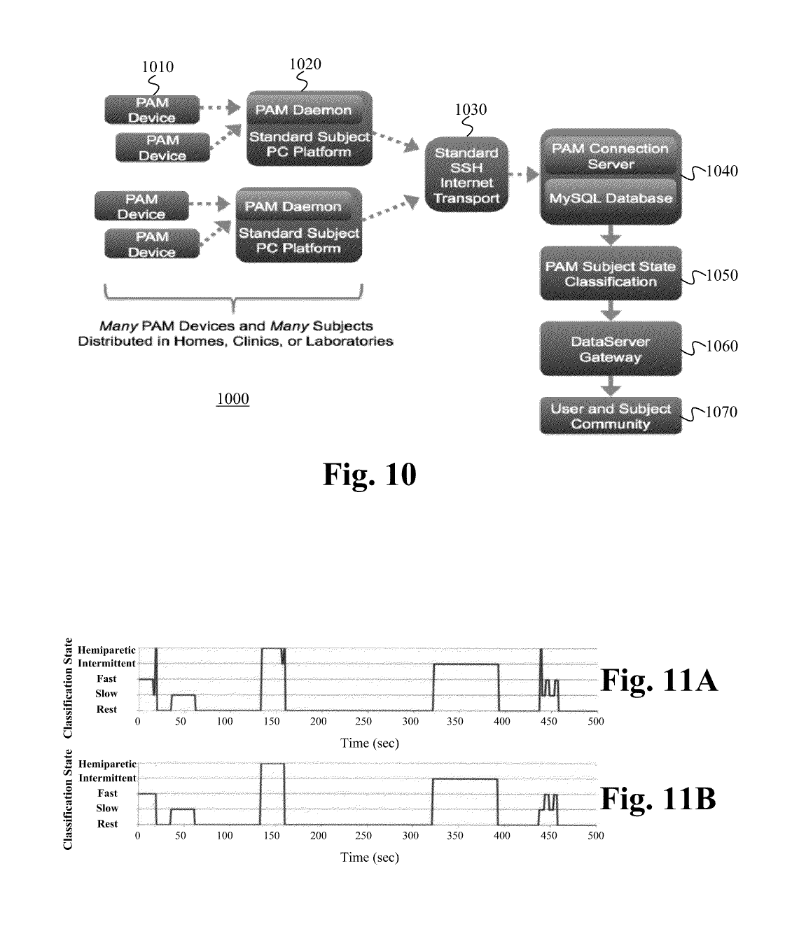

FIG. 10 illustrates a PAM system architecture 1000 in accordance with some embodiments of the present invention. The system 1000 includes data transport, data archiving, data processing with subject state classification, and data delivery. In some embodiments, data transport for all devices and interfaces is protected with established standard solutions. In some embodiments, each device uses established standards for authentication as well as for secure data transport.

Subject data is acquired by, preferably wearable, PAM devices 1010. In some embodiments, one or more PAM devices 1010 being used by a single subject are communicatively coupled to a single computer 1020, such as a computer comprising a standard subject PC platform. In some embodiments, a PAM daemon runs on the computer and transmits data obtained from the one or more measurement devices 110 to a server 1040 via communication means 1030, such as a standard SSH internet transport. In some embodiments, the computer 1020 transmits the data unmodified from the form it is received in from the measurement device 1010. In some embodiments, the computer 1020 processes and modifies the form of the data received from the measurement device 1010 before it transmits the data to the server 1040. In some embodiments, the server comprises a database, such as a MySQL database, which is used to archive the received data (or a processed version of the received data). At 1050, the server uses algorithms to analyze and classify the state of the subject using the PAM device 1010. A user (e.g., subject, guardian, physician) 1070 uses a server gateway 1060, such as the DataServer gateway, to access results provided by the server.

In some embodiments, the architecture 1000 is additionally developed at certain levels to meet the needs of the research community. The modular PAM architecture of the present invention provides replaceable and reconfigurable subject state classification systems, so that investigators can evaluate signal processing algorithms and rules. New data and demands will drive modifications and entirely new features. In some embodiments, each component of the repository is open source software, and developmental projects associated with the program are shared with the public.

In some embodiments, the present invention is used for classification studies that deal with more quantitative aspects of the quality of identified movement patterns. For example, FIGS. 11A-B show the results of gait classification using bilateral distal leg sensors for a subject executing 5 behaviors (from left to right)—walking with a normal gait at two speeds, executing a right hemiparetic walking pattern, intermittent normal walking with momentary pauses in motion, and then variable fast and slow normal walking patterns. FIG. 11B shows the actual measured gait. The autonomous classification shown in FIG. 11A is accurate in each case.

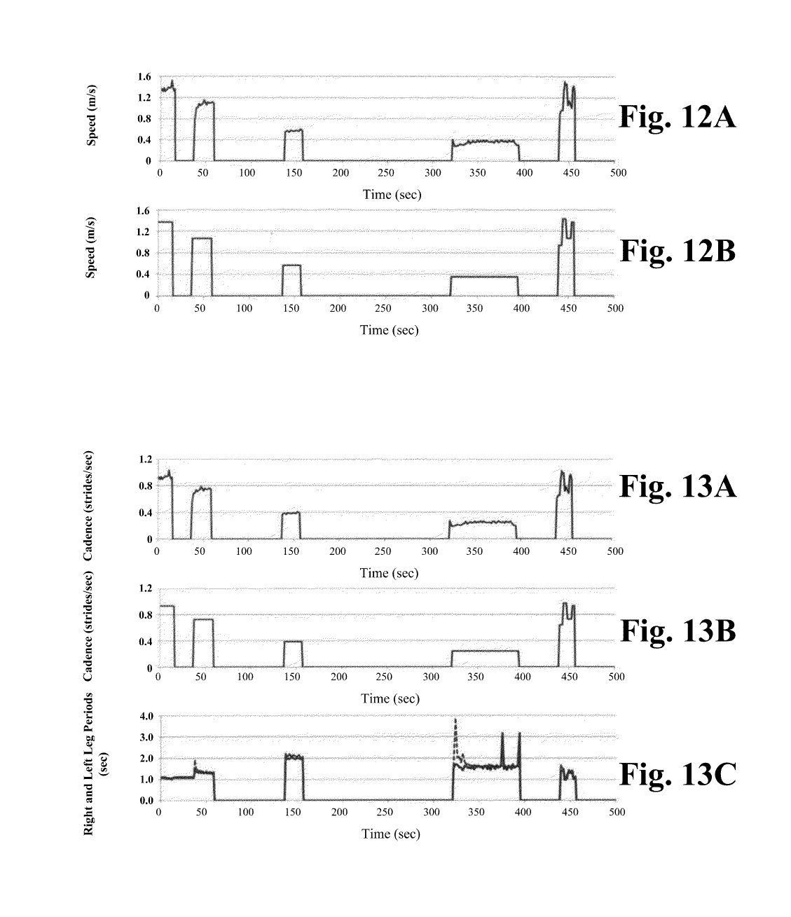

FIGS. 12-B provide the corresponding walking speeds from FIGS. 11A-B, with the result of automatic measurement of walking speed, shown in FIG. 12A, compared to the actual measured speed, shown in FIG. 12B.

FIGS. 13A-B show cadence measurements for each behavior in FIGS. 11A-12B. FIG. 13A shows the results of automatic measurement, and FIG. 13B shows actual observed cadence. FIG. 13C shows the ratio of right to left leg stride period.

Thus, these exemplar classifications are very accurate. The accelerometry signals, especially when acquired from multiple limbs and in all 3 axes, and perhaps when examined with other sensor and clinical data, also offer a rich resource for detailed analyses of the quality of movements—speed, precision, forces, calculation of joint moments, identification of compensatory movements, and alterations that occur when subjects are faced with new environmental challenges.

The PAM system is constructed in a modular design so that each module can be edited or applied in sequence. For example, in some embodiments, a server-side motion feature library is applied to select the best combination of features for a specific application or even for a specific subject. Furthermore, in some embodiments, the training procedure that creates a model relating feature space to subject classes is individualized, so data is as easily shared or researched as contributing investigators wish. Modularity also enables further development to collect and synchronize accelerometers, gyroscopes, GPS, video, voice, and other markers of activity as the need arises.

In addition to continued studies for classification, the PAM system preferably includes a library of sequence search algorithms that enable an automated identification of patterns or events with different properties. For example, two of the most common properties are patterns that repeat cyclically and events/abnormalities that are not common for a given sensor data set, but complex enough not to be considered noise. Both of these types of sequence search algorithms are useful in automatically analyzing collections of sensor data with unknown parameters or events. These algorithms can be effective in finding features for a given subject state that are otherwise not known or are difficult to identify visually (i.e., classification tasks). Further development should give investigators additional search tools and data sets.

In some embodiments, valuable additions to assistive devices could lead to an increase in activity. For example, the SmartCane system was developed by inventors M. Batalin and W. Kaiser with low-cost, long operating embedded computing systems. The low-power wireless interface on the SmartCane system permits it to integrate with wearable sensors, standard handheld personal wireless devices, the Internet, and remote services such as a call center in case of a fall. In some embodiments, the diverse set of low-cost microsensors incorporated into the cane enables the measurement of motion, rotation, force, strain, and impact signals. In some embodiments, both assistive devices and exercise equipment sensors are further integrated with PAM technology.

In some embodiments, the present invention provides immediate feedback about performance, as the subject exercises or simply moves about, as well as summary feedback about exercise or other activity. In some embodiments, feedback to subjects is provided on a PDA or computer screen or as an emailed message, providing accessible charts of overall progress in therapy. An interface program allows the clinician to prescribe rehabilitation exercises to patients. In some embodiments, the interface includes a list of all possible exercises for the patient to perform so that the clinician can quickly and easily progress the type, number of repetitions and number of sets or variations for the patient's rehabilitation.

In some embodiments, the present invention is used to develop physiological and medical knowledge management systems that are integrated with telecommunications and information processing to enhance decision-making, training, improve the development and delivery of rehabilitation treatments, and redefine the possibilities for compliance and outcome measures.

The present invention has been described in terms of specific embodiments incorporating details to facilitate the understanding of principles of construction and operation of the invention. Such reference herein to specific embodiments and details thereof is not intended to limit the scope of the claims appended hereto. It will be readily apparent to one skilled in the art that other various modifications can be made in the embodiment chosen for illustration without departing from the spirit and scope of the invention as defined by the claims.

What is claimed is:

1. A system for monitoring patient activity comprising:
at least one measurement device configured to provide data related to a patient's physical activity; and
a server configured to make an inference regarding the patient's physical activity based on data provided by the at least one measurement device.

2. The system of claim 1, wherein the inference is a determination of a type of physical activity.

3. The system of claim 1, wherein the at least one measurement device is configured to provide the data related to the patient's physical activity from a location remote from the server.

4. The system of claim 1, wherein the at least one measurement device is configured to be worn by the patient or carried in the patient's pocket.

5. The system of claim 1, wherein the at least one measurement device is configured to transmit the data related to the patient's physical activity via wireless communication.

6. The system of claim 1, wherein the at least one measurement device comprises two or more measurement devices each configured to provide data related to the patient's physical activity.

7. The system of claim 1, wherein the at least one measurement device comprises a triaxial accelerometer, a microgyroscope, or a pressure sensor.

8. The system of claim 1, wherein the at least one measurement device is configured to automatically take repeated data samples.

9. The system of claim 1, wherein the server is configured to infer the probability of a patient being in an activity state based on the data provided by the at least one measurement device.

10. The system of claim 1, wherein the server is configured to make the inference based on a combination of data obtained from different measurement devices corresponding to different parts of the patient's body.

11. The system of claim 10, wherein the data in the combination of data is based on samples being taken simultaneously by the different measurement devices.

12. The system of claim 1, wherein the server is configured to make the inference by applying Bayesian Sensor Fusion analysis in making the inference.

13. The system of claim 12, wherein the server is configured to apply a naive Bayer classifier model to infer the probability of a patient state vector given a feature vector.

14. The system of claim 1, wherein the server is configured to use a Fourier transform in processing data provided by the at least one measurement device in a time domain to extract frequency spectral components.

15. The system of claim 14, wherein the server is configured to use a Fast Fourier transform.

16. The system of claim 1, wherein the server is configured to make the inference by using a fundamental frequency component and spectrum energy.

17. The system of claim 1, wherein the server is configured to make the inference by applying one or more motion recognition algorithms

18. The system of claim 1, wherein the server is configured to make the inference by applying one or more state classification algorithms to make the inference.

19. The system of claim 1, wherein the server is configured to archive and retrieve the data provided by the at least one measurement device and the inferences.

20. A method of monitoring patient activity, the method comprising:
a server receiving data related to a patient's physical activity, wherein the data is based on one more samples from at least one measurement device; and
the server making an inference regarding the physical activity based on the received data

21. The method of claim 20, wherein the inference is a determination of a type of physical activity.

22. The method of claim 20, wherein the server is located remotely from the at least one measurement device.

23. The method of claim 20, wherein the step of the server receiving the data is preceded by a step of the at least one measurement device taking one or more samples of the patient's physical activity.

24. The method of claim 23, wherein the at least one measurement device is worn by the patient or carried in the patient's pocket when the one or more samples are taken.

25. The method of claim 23, wherein the at least one measurement device transmits the data via a wireless communication.

26. The method of claim 23, wherein the at least one measurement device comprises a triaxial accelerometer, a microgyroscope, or a pressure sensor.

27. The method of claim 23, wherein the at least one measurement device automatically takes repeated data samples.

28. The method of claim 20, wherein the server infers the probability of a patient being in an activity state based on the data provided by the at least one measurement device.

29. The method of claim 20, wherein the server makes the inference based on a combination of data obtained from different measurement devices corresponding to different parts of the patient's body.

30. The method of claim 29, wherein the data in the combination of data is based on samples being taken simultaneously by the different measurement devices.

31. The method of claim 20, wherein the server applies Bayesian Sensor Fusion analysis in making the inference.

32. The method of claim 31, wherein the server applies a naive Bayer classifier model to infer the probability of a patient state vector given a feature vector.

33. The method of claim 20, wherein the server uses a Fourier transform in processing data provided by the at least one measurement device in a time domain to extract frequency spectral components.

34. The method of claim 33, wherein the server uses a Fast Fourier transform.

35. The method of claim 20, wherein the server makes the inference by using a fundamental frequency component and spectrum energy.

36. The method of claim 20, wherein the server makes the inference by applying one or more motion recognition algorithms

37. The method of claim 20, wherein the server makes the inference by applying one or more state classification algorithms

38. The method of claim 20, further comprising the server archiving the received data and the inferences for subsequent retrieval.

39. A program storage device readable by a machine, tangibly embodying a program of instructions executable by the machine to perform a method of monitoring patient activity, the method comprising:
making an inference regarding a patient's physical activity based on data related to the patient's physical activity, wherein the data is based on one more samples from at least one measurement device.

40. The device of claim 39, wherein the inference is a determination of a type of physical activity.

41. The device of claim 39, wherein making the inference comprises inferring the probability of a patient being in an activity state based on the data provided by the at least one measurement device.

42. The device of claim 39, wherein the inference is based on a combination of data obtained from different measurement devices corresponding to different parts of the patient's body.

43. The device of claim 42, wherein the data in the combination of data is based on samples that have been taken simultaneously by the different measurement devices.

44. The device of claim 39, wherein making the inference comprises applying Bayesian Sensor Fusion analysis.

45. The device of claim 44, wherein the method further comprises applying a naïve Bayer classifier model to infer the probability of a patient state vector given a feature vector.

46. The device of claim 39, wherein the method further comprises using a Fourier transform in processing data provided by the at least one measurement device in a time domain to extract frequency spectral components.

47. The device of claim 46, wherein the method further comprises using a Fast Fourier transform in processing data

48. The device of claim 39, wherein making the inference comprises using a fundamental frequency component and spectrum energy.

49. The device of claim 39, wherein making the inference comprises applying one or more motion recognition algorithms

50. The device of claim 39, wherein making the inference comprises applying one or more state classification algorithms

51. The device of claim 39, wherein the method further comprises archiving the received data and the inferences for subsequent retrieval.

52. A system for training a model for monitoring patient activity, the system comprising a server configured to:
extract features from training data;
cluster the extracted features into a discrete feature space; and
perform a maximum likelihood estimation for the discrete feature space to construct a maximum likelihood model.

53. The system of claim 52, wherein the server is configured to cluster the extracted features using Gaussian cluster discretization.

54. The system of claim 52, wherein the server is further configured to correlate features with different states of activity for a patient.

55. A method of training a model for monitoring patient activity, the method comprising:
extracting features from training data;
clustering the extracted features into a discrete feature space; and
performing a maximum likelihood estimation for the discrete feature space to construct a maximum likelihood model.

56. The method of claim 55, wherein clustering the extracted features comprises performing Gaussian cluster discretization.

57. The method of claim 55, further comprising the step of correlating features with different states of activity for a patient.

58. A program storage device readable by a machine, tangibly embodying a program of instructions executable by the machine to perform a method of training a model for monitoring patient activity, the method comprising:
extracting features from training data;
clustering the extracted features into a discrete feature space; and
performing a maximum likelihood estimation for the discrete feature space to construct a maximum likelihood model.

59. The device of claim 58, wherein clustering the extracted features comprises performing Gaussian cluster discretization.

60. The device of claim 58, wherein the method further comprises the step of correlating features with different states of activity for a patient.
